(19) United States
(12) Patent Application Publication
(10) Pub. No.: US 2002/0187919 A1
(43) Pub. Date: Dec. 12, 2002

(54) Methods for preventing pressure induced apoptotic neural cell death

(76) Inventor: Minas Theodore Coroneo, Randwick (AU)

Correspondence Address: MARSHALL, GERSTEIN & BORUN, 6300 SEARS TOWER, 233 SOUTH WACKER, CHICAGO, IL, 60606-6357, US

(21) Appl. No.: 10/084,604

(22) Filed: Feb. 27, 2002

Related U.S. Application Data
(63) Continuation of application No. 09/649,643, filed on Aug. 29, 2000, now abandoned.

(51) Int. Cl. A61K 31/00

(52) U.S. Cl. 514/1

(57) ABSTRACT

Compositions and methods for protecting neural tissue from pressure induced apoptotic cell death which comprises administering to a subject in need of such treatment at least one company which blocks the effect of pressure on neuronal cells.

### FILED OF THE INVENTION

This invention is concerned with methods and compositions for protecting neural tissue from cell death, more particularly, apoptotic cell death associated with pressure. In a further aspect the invention is concerned with methods and compositions for the treatment of glaucoma, elevated brain pressure, and peripheral nerve damage associated with elevated pressure.

### BACKGROUND OF THE INVENTION

Neuronal tissue or nerve cell death is a major medical problem in human society. Neuronal cell death in the eye may lead to blindness. Glaucoma, a principal cause of neural cell apoptotic death in the eye and a principal cause of adult blindness. It is the third major cause of visual loss in the elderly, affecting approximately 3% of the population over 50.

Neuronal cell death is associated with a range of other medical conditions. These include hydrocephalus, and other brain/skull diseases or injuries. Brain neurone cell death may result in mental impairment, loss of motor functions and the like.

Peripheral nerve damage from traumatic injury or surgical complications, for example, in the spine, feet and hands may cause apoptotic cell death. In the spinal column where spinal bones may press upon a nerve trunk causing nerve cell death. Bone and connective tissue pressures in peripheral tissue such as the wrists may cause apoptotic neural cell death and consequent lack of feeling and/or motor movement.

Morphologically apoptosis is characterised by progressive condensation of the cytoplasma nucleus, followed by fragmentation and phagocytosis by other cells (Majino and Joris (1995) Am Pathol 146: 3-15).

Although there are some known inhibitors of apoptosis, there are no effective therapeutic agents for the treatment of apoptotic neuronal cell death. This reflects the lack of understanding of the precise mechanisms involved.

In relation to glaucoma, there are now a number of agents which reduce eye pressure, with mixed success. The mechanism of action of such agents is controversial and unclear. Glaucoma remains one of the major causes of blindness in human society (accounting for approximately 15% of cases of blindness).

Similarly, apoptotic neural cell death associated with wide range of conditions, such as hydrocephalus, spinal compression, and peripheral neuronal cell pressure mentioned above, remain significant problems, with no effective (non-surgical) therapeutic agents being available.

### SUMMARY OF THE INVENTION

In a first aspect of this invention, there is provided a method for protecting neural tissue from pressure induced apoptotic cell death which comprises contacting the cells with at last one compound which blocks the effect of pressure on the cells.

In another aspect there is provided a method of protecting neural tissue from pressure induced apoptotic cell death which comprises administering to a subject in need of such treatment at least one compound which blocks the effects of pressure on neuronal cells.

In another aspect there is provided a method of protecting neural tissue from pressure induced apoptotic cell death which comprises administering to a subject in need of such treatment at least one compound which blocks stretch-activated channels (either directly or indirectly) or other pressure sensitive cellular mechanisms in neuronal cells.

In another aspect of the invention there is provided a method for the treatment of glaucoma which comprises administering to a subject in need of such treatment an effect amount of a composition which blocks the effect of pressure on neural cells in the eye.

In another aspect of the invention there is provided a method for the treatment of glaucoma which comprises administering to a subject in need of such treatment a compound which blocks stretch-activated channels (either directly or indirectly) or other pressure sensitive cellular mechanisms in eye neuronal cells.

In a another aspect of the invention there is provided a method for the treatment of the effects of elevated brain pressure which comprises administering to a subject in need of such treatment a compound which blocks stretch-activated channels (either directly or indirectly) or other pressure sensitive cellular mechanisms in brain neuronal cells.

In a another aspect of the invention there is provided a method for the treatment of peripheral nerve damage which comprises administering to a subject in need of such treatment a compound which blocks stretch-activated channels (either directly or indirectly) or other pressure sensitive cellular mechanisms in said peripheral nerve cells.

In a further aspect of the invention there is provided a composition for protecting neural tissue from pressure induced apoptotic cell death which comprises an effective amount of at least one compound which blocks stretch-activated channels (either directly or indirectly) or other pressure sensitive cellular mechanisms in neuronal cells, optionally in association with one or more pharmaceutically acceptable carriers or excipients.

In a further aspect of the invention there is provided a composition for the treatment of glaucoma which comprises at least one compound which blocks stretch-activated channels (either directly or indirectly) or other pressure sensitive cellular mechanisms in eye neuronal cells, optionally in association with one or more pharmaceutically acceptable carriers or excipients.

In a further aspect of the invention there is provided a composition for the treatment of elevated brain pressure which comprises at least one compound which blocks stretch-activated channels (either directly or indirectly) or other pressure sensitive cellular mechanisms in neuronal cells, optionally in association with one or more pharmaceutically acceptable carriers or excipients.

In a further aspect of the invention there is provided a composition for the treatment of peripheral nerve damage which comprises at least one compound which blocks stretch-activated channels (either directly or indirectly) or other pressure sensitive cellular mechanisms in neuronal cells, optionally in association with one or more pharmaceutically acceptable carriers or excipients.

### DETAILED DESCRIPTION OF THE INVENTION

This invention provides methods and compositions for protecting neural tissue from pressure induced apoptotic cell death. The invention is based on the surprising finding that elevated pressure on neuronal cells induces apoptotic cell death. The invention is also based on the unexpected finding that compounds which block stretch-activated channels (either directly or indirectly) or other pressure sensitive cellular mechanisms in neuronal cells protect the neuronal cells against pressure induced apoptotic cell death.

The effects of pressure on neuronal cells may be blocked though the use of compounds which block stretch-activated channels (either directly or indirectly) or other pressure sensitive cellular mechanisms in neuronal cells, such as those associated with the cell membrane and/or those associated with intracellular membranes, such as those of mitochondria and T tubules.

In another aspect the invention is concerned with the method of protecting neural tissue from pressure induced apoptotic cell death which comprises administering to a subject in need of such treatment at least one compound which blocks stretch-activated channels (either directly or indirectly) or other pressure sensitive cellular mechanisms in said neuronal cells.

Stretch-activated channels have been described by various authors, and may be regarded as being associated with mechanoelectric transduction (see Zeng et al (2000) Heart and Circulatory Physiology 278 (2): H548). Stretch-activated channels (SACs) are found in a variety of cells including cardiomyocytes (see Hu and Sachs (1996) J Membr Biol 154: 205-216).

Blocking of stretch-activated channels may be measured according to conventional physiological techniques, such as by voltage clamp recordings from isolated cells subject to membrane stretching, for example resulting from increased pressure or induced physical stretching, such as subjecting isolated cells to controlled strain such as longitudinal stretch. Under these conditions, stretch-activated channels may be measured by elicited electrical current. The elicited current may represent inward cationic currents such as described by Zeng et al (2000) Heart and Circulatory Physiology 278 (2): H548. Agents which block stretch-activated channels bind to or are associated with the channels so as to reduce or abolish the elicited currents. Preferably stretch induced currents are reduced by the compounds, for example, by between 10% and 100%, such as 10% and 90%, 10% and 80%, 10% and 70%, 10% and 60%, 10% and 50%, 10% and 40%, 10% and 30%, 10% and 20%.

In another aspect this invention is concerned with the method of protecting neural tissue from pressure induced apoptotic cell death which comprises administering to a subject in need of such treatment at least one compound which blocks stretch-activated channels (either directly or indirectly) or other pressure sensitive cellular mechanisms in neuronal cells.

In another aspect of the invention there is provided a method for the treatment of glaucoma which comprises administering to a subject in need of such treatment at least one compound which blocks stretch-activated channels (either directly or indirectly) or other pressure sensitive cellular mechanisms in neuronal cells.

In another aspect of the invention there is provided a method for the treatment of elevated brain pressure which comprises administering to a subject in need of such treatment at least one compound which blocks stretch-activated channels (either directly or indirectly) or other pressure sensitive cellular mechanisms in brain neuronal cells.

In a another aspect of the invention there is provided a method for the treatment of peripheral nerve damage which comprises administering to a subject in need of such treatment at least one compound which blocks stretch-activated channels (either directly or indirectly) or other pressure sensitive cellular mechanisms in peripheral nerve cells.

In a further aspect of the invention there is provided a composition for protecting neural tissue from pressure induced apoptotic cell death which comprises at least one compound which blocks stretch-activated channels in neuronal cells, optionally in association with one or more pharmaceutically acceptable carriers or excipients.

In a further aspect of the invention there is provided a composition for the treatment of glaucoma which comprises at least one compound which blocks stretch-activated channels (either directly or indirectly) or other pressure sensitive cellular mechanisms in neuronal cells, optionally in association with one or more pharmaceutically acceptable carriers or excipients.

In a further aspect of the invention there is provided a composition for the treatment of elevated brain pressure which comprises at least one compound which blocks stretch-activated channels (either directly or indirectly) or other pressure sensitive cellular mechanisms in neuronal cells, optionally in association with one or more pharmaceutically acceptable carriers or excipients.

In a further aspect of the invention there is provided a composition for the treatment of peripheral nerve damage which comprises a protectant which blocks stretch-activated channels (either directly or indirectly) or other pressure sensitive cellular mechanisms in neuronal cells, optionally in association with one or more pharmaceutically acceptable carriers or excipients.

Compounds which block the apoptotic effect of pressure on neuronal cells, and in turn which may block stretch-activated channels (see Hamill O, McBride D W, Pharmacol Rev 1996; 48: 231-252) include: gandolinium (Gd³⁺), a lanthanide; pyrazine-carboxamides such as amiloride and its analogues (as described by Kleyman and Cragoe (1998) J Membr Biol 105:1-21 and Kleyman and Cragoe (1990) Methods Enzymol 191:739-754); aminoglycoside antibiotics (such as verdamycin, gentamycin, sisomycin, streptomycin, dihydrostreptomycin, netilmycin, amikacin, ribostamycin, dibekacin, kanamycin; other blockers including: Na channel blockers, Ca channel blockers, K channel blockers; Ca ions; protons; aluminium ions; tubocurarine; halothane and other inhalational anaesthetics; quinine; positively charged medium to long-chained fatty acids and fatty acid analogues (such as arachodonic acid, linoleic acid, γ-linoleic acid, docsahexanoic acid, oleic acid, tetradecanesulphonate, and myristic acid); integrin-blocking peptides and antibodies, cisplatin; tarantula spider venom; colchicine and vinblastine. At least one active compound is used in the compositions and methods of this invention. For example, two or more compounds may be used in combination. Such combinations may involve synergistic interactions. The effects may occur directly on the stretch-activated channels (SAC) or indirectly via actions on the cytoskelton, extracellular matrix or on mechanosensitive enzymes (phospholipase A2 and phospholipase C). Inhibitors of these mechanisms are also within the scope of this invention.

The invention is not limited to the aforementioned compounds and includes any compound which blocks the apoptotic effects of pressure on neuronal cells. Suitable compounds can be readily identified by testing apoptotic protecting activity under pressure, whether, for example, under atmospheric or hydrostatic pressure or such as by physical stretching of cells. Where neuronal cells are subjected to elevated pressure, such as 100 mm Hg for two hours or more, pressure induced apoptotic cell death occurs, as can be determined by apoptosis assays (see Agar A et al J Neurosci Res 2000; 60: 495-503). Compounds which inhibit such cell death may be used in this invention.

Similarly, compounds which block stretch-activated channels in neuronal cells can be readily-identified by conventional physiological techniques, such as patch/voltage clamp recordings from isolated neuronal cells subject to elevated pressure as described above (Zeng et al (2000) Heat and Circulatory Physiology 278(2):H548). Compounds which block elicited currents may be used in this invention.

Compositions according to the invention may be formulated with standard buffers, excipients, carriers, diluents and the like. Examples of carriers include: water, physiologically saline, isotonic solutions containing dextrose, glycerol or other agents conferring isotonicity, lower alcohols, vegetable oils, polyethylene glycol, glycerol triacetate and other fatty acid glycerides. Examples of other carriers which may be used include cream forming agents, gel forming agents, and the like, compounding and tabletting agents. Excipients include buffers, stabilisers, emulsion forming agents, colouring compounds, salts, amino acids, antibiotics and other anti-bacterial compounds chelating agents and the like. More than one excipient and carrier may be used.

The amount of compounds used to protect neural tissue from pressure induced apoptotic cell death will depend upon various factors including the neural tissue to be treated, such as that in the eye, in the brain, or in peripheral tissue such as in the hand, leg, foot, fingers, oral cavity, nose or ear, the manner of delivery, the severity of the condition being treated, and the judgement of the prescribing physician. By way of example, compounds of the invention may be delivered as a solution for installation, such as an eye drop, ear drop, nose drop; an injectible sterile subcutaneous or intravenous solution; in the form of a tablet, capsule, suppository, dragee; or in the form of a transdermal composition; all of which are well known in the pharmaceutical field and described for example in Remington's Pharmaceutical Sciences Mack Publishing Company, Philadelphia. Generally, the concentration of active agents, which may be regarded as therapeutically effective, will be in the order of 0.001 m to 500 mM, such as from 0.1 m to 100 m, 50 m to 100 m, 100 m to 500 m, 500 m to 1 mM, or 1 mM to 500 mM.

In relation to the treatment of glaucoma compositions of the invention may be administered to the eye, such as by way of eye drop or intraocular injection or as a systemic medication such as a tablet etc.

The present invention in one of its aspects represents a significant advance in relation to the treatment of glaucoma. Theories of glaucoma pathogenesis to date are controversial and unclear. Whilst elevated pressure in the eye is a characteristic of glaucoma, the ways in which retinal ganglion cell death is mediated, and may be prevented, are unknown. The inventor's work indicates that pressure alone may be the stimulus for apoptosis in neuronal cells, both in culture and in vivo. Blocking the apoptotic effect of pressure on neuronal cells, such as by inhibiting stretch-activated channels, provides therapeutic outcomes.

Compositions for the treatment of glaucoma may be administered to a subject one or more times per day, on or alternative days as a single administration or on a weekly basis. It is preferred that the compositions are administered to the eye for the treatment of glaucoma on a daily basis, generally from 1 to 3 times per day, such as at 5 to 8 hour intervals.

In the treatment of elevated brain pressure, such as that due to hydrocephalus, compounds of the invention may be formulated by conventional means known in the art so as to cross the blood-brain barrier. Such compositions may be administered parenterally or non-parenterally as described above, such as by way of oral administration in the form of a capsule, table or the like, rectal or vaginal administration, intravenous administration or intramuscular administration.

In the treatment of pressure induced apoptotic neuronal cell death in peripheral nerves, administration of the compounds of the invention will depend upon the site of the neurons/condition being treated. By way of example, increased neuronal cell pressure in the spine may be treated by way of transdermally active compositions, intramuscular injection, intralumbar injection, intravenous administration, oral administration, rectal administration or inhalation administration.

1. A method of protecting neural tissue from pressure induced apoptotic cell death which comprises administering to a subject in need of such treatment at least one compound which blocks the effects of pressure on neuronal cells.

2. A method of protecting neural tissue from pressure induced apoptotic cell death which comprises administering to a subject in need of such treatment at least one compound which blocks stretch-activated channels (either directly or indirectly) or other pressure sensitive cellular mechanisms in neuronal cells.

3. A method for the treatment of glaucoma which comprises administering to a subject in need of such treatment at least one compound which blocks stretch-activated channels (either directly or indirectly) or other pressure sensitive cellular mechanisms in eye neuronal cells.

4. A method for the treatment of the effects of elevated brain pressure which comprises administering to a subject in need of such treatment at least one compound which blocks stretch-activated channels (either directly or indirectly) or other pressure sensitive cellular mechanisms in brain neuronal cells.

5. A method for the treatment of peripheral nerve damage which comprises administering to a subject in need of such treatment at least one compound which blocks stretch-activated channels (either directly or indirectly) or other pressure sensitive cellular mechanisms in neuronal cells.

6. A composition for protecting neural tissue from pressure induced apoptotic cell death which comprises at least one compound which blocks the effects of pressure on neuronal cells, optionally in association with one or more pharmaceutically acceptable carriers or excipients.

7. A composition for the treatment of glaucoma which comprises at least one compound which blocks stretch-activated channels (either directly or indirectly) or other pressure sensitive cellular mechanisms in neuronal cells, optionally in association with one or more pharmaceutically acceptable carriers or excipients.

7. A composition for the treatment of elevated brain pressure which comprises at least one compound which blocks stretch-activated channels (either directly or indirectly) or other pressure sensitive cellular mechanisms in brain neuronal cells, optionally in association with one or more pharmaceutically acceptable carriers or excipients.

8. A composition for the treatment of peripheral nerve damage which comprises at least one compound which blocks stretch-activated channels in peripheral neuronal cells (either directly or indirectly) or other pressure sensitive cellular mechanisms, optionally in association with one or more pharmaceutically acceptable carriers or excipients.
